(19) United States
(12) Patent Application Publication
(10) Pub. No.: US 2026/0199012 A1
(43) Pub. Date: Jul. 16, 2026

(54) DEVICES SUITABLE FOR USE IN PERFORMING OTOLARYNGOLOGY PROCEDURES AND KIT COMPRISING SUCH DEVICES

(71) Applicant: Barry Kriegsman, Philadelphia, PA (US)

(72) Inventor: Barry Kriegsman, Philadelphia, PA (US)

(21) Appl. No.: 19/452,067

(22) Filed: Jan. 16, 2026

Related U.S. Application Data
(60) Provisional application No. 63/745,973, filed on Jan. 16, 2025.

(51) Int. Cl. A61B 18/22; A61B 1/00; A61B 1/267; A61B 18/00; A61B 18/20

(52) U.S. Cl. CPC A61B 18/22; A61B 1/00071; A61B 1/00128; A61B 1/00147; A61B 1/267; A61B 2018/00172; A61B 2018/00184; A61B 2018/00327; A61B 2018/00982; A61B 2018/202; A61B 2018/20359; A61B 2018/2283

(57) ABSTRACT

System suitable for use in performing an otolaryngology procedure. In one embodiment, the system includes a laryngoscope, an endoscope assembly, and a laser beam targeting assembly. The endoscope assembly, in turn, includes an endoscope and an endoscope positioning device. The endoscope positioning device includes a sheath and a frame, the sheath being mountable on the frame. The sheath includes a jacket insertable over the insertion tube of the endoscope and a projection extending outwardly from the jacket. The frame, which may be removably secured to the laryngoscope with a clip, includes a support having a deployable stop that engages the projection and delimits axial movement of the sheath. The laser beam targeting assembly includes a platform that may be removably secured to the laryngoscope. The platform includes a first docking port for receiving a laser handpiece and a second docking portion for receiving a steerable mirror on a wand.

### CROSS-REFERENCE TO RELATED APPLICATIONS

The present application claims the benefit under 35 U.S.C. 119(e) of U.S. Provisional Patent Application No. 63/745,973 , inventor Barry Kriegsman, filed Jan. 16, 2025, the disclosure of which is incorporated herein by reference.

### BACKGROUND OF THE INVENTION

The present invention relates generally to devices suitable for use in performing otolaryngology procedures and relates more particularly to novel devices of this type and to a kit comprising such devices.

Otolaryngology is a specialized field of medicine that focuses on the throat including the larynx (i.e., the voice box) and nearby areas of interest. Otolaryngology procedures are often performed with the aid of a particular type of medical device known as a laryngoscope. Typically, a laryngoscope is constructed to include a distal portion (i.e., a blade), which is insertable into the mouth of a patient, and a proximal portion (i.e., a handle), which is designed to remain external to the patient and which may be used to manipulate and/or to immobilize the blade of the laryngoscope relative to the patient. The blade and the handle of the laryngoscope are typically oriented at an angle relative to one another, and the blade of the laryngoscope typically includes a longitudinal channel extending from its proximal end to its distal end. This longitudinal channel may be used to receive one or more instruments, which may include, for example, (i) an endoscope, which may be used for visualization of an area of interest typically located distally beyond the blade of the laryngoscope, (ii) a surgical laser, which may be used for treatment (e.g., ablation, resection, etc.) of an area of interest typically located distally beyond the blade of the laryngoscope, and (iii) one or more other types of diagnostic or surgical instruments (e.g., an aspirator, a retractor, etc.).

Otolaryngology procedures involving the use of a surgical laser are typically performed according to one of two types of techniques. According to one such technique, which is commonly referred to in the field as microscopic-guided laser surgery or trans-oral laser microsurgery, the blade of a laryngoscope is inserted into the mouth of a patient, and a microscope is positioned proximal to the blade of the laryngoscope. The microscope is optically aligned with the longitudinal channel of the blade of the laryngoscope so that a surgeon may view an area of interest in the patient simply by looking into the microscope and adjusting the magnification of the microscope as needed. Once an area of interest has been identified and magnified to an extent such that the surgeon no longer needs to adjust the microscope, the surgeon can continue to use the microscope for viewing the area of interest but has both hands free for the procedure to be performed. Thus, the surgeon may use one hand to operate a micromanipulator, i.e., a device used to control the operation of a surgical laser that is located external to the patient but whose output is directed into and through the longitudinal channel of the laryngoscope blade. (Instead of using one hand to control a micromanipulator, the surgeon may alternatively use one hand to insert a handpiece carrying an optical fiber, coupled to an external surgical laser, into the longitudinal channel of the laryngoscope blade.) In addition, the surgeon may use another hand to control an additional instrument, such as an aspirator or a retractor, also inserted into the longitudinal channel of the laryngoscope blade.

Unfortunately, microscopic-guided laser surgery suffers from certain shortcomings. For one thing, using a microscope that is positioned external to the laryngoscope (and, thus, a considerable distance from the area of interest being viewed) often leads to an image of the area of interest that is of sub-optimal quality and stability. Moreover, using a microscope in this fashion for visualization of the area of interest may result in blurring or obscuring of the image due to the positioning of another instrument distal to the microscope. Additionally, the micromanipulator may only direct the laser beam onto targets that are in the line of sight of the surgeon/microscope, which can limit access to certain areas beyond the exposure of the laryngoscope.

Another type of technique for performing laryngology procedures involving a surgical laser is commonly referred to in the field as endoscopic laser surgery. In endoscopic laser surgery, the blade of a laryngoscope is inserted into the mouth of a patient, and a surgeon views an area of interest in the patient using an endoscope that is inserted into the longitudinal channel of the laryngoscope blade. The endoscope typically includes one or more optical fibers coupled to a light source for illuminating an area of interest and also includes imaging optics and an optical fiber for delivering an image of the illuminated area to the proximal end of the endoscope. A surgeon may view the image by looking directly through an eyepiece located at the proximal end of the endoscope; alternatively, the eyepiece may be operatively coupled to a monitor, thereby enabling the surgeon to view the image of the illuminated area as it is displayed on the monitor. Typically, the surgeon holds and positions the endoscope in the patient using one hand. Consequently, the surgeon only has one additional hand (i) for inserting another instrument, such as a surgical laser handpiece, into the longitudinal channel of the laryngoscope blade and (ii) for operating said instrument.

As can readily be appreciated, limiting the surgeon to the use of only one free hand (i.e., the hand not holding the endoscope) to perform a procedure is disadvantageous in many respects. For example, where the free hand of the surgeon is being used to operate a surgical laser handpiece, it may be necessary or desirable for suction to be applied, using an aspirator, concurrently with the operation of the surgical laser. However, since the surgeon is already using both hands, one for the endoscope and the other for the surgical laser handpiece, the surgeon does not have a free hand to operate an aspirator. Consequently, the operation of an aspirator is typically left to an assistant, who is not in as favorable a position ergonomically as the surgeon, to perform accurate, controlled movements with the aspirator.

One approach that has been taken to obviate the need for the surgeon to continuously hold the endoscope in position in the laryngoscope is to use a laryngoscope having two parallel channels, namely, a main channel and a side channel. The main channel is typically used to receive instruments like a surgical laser handpiece and/or an aspirator, and the side channel is used to receive the endoscope. Such a side channel often includes a distal portion of reduced size (sometimes referred to as an endoscope carrier) that delimits distal movement of the endoscope in the side channel. (Since the patient is typically lying supine during the procedure, distal movement is typically down (i.e., in the same direction as gravity) to a defined depth.) As a result, the endoscope can only be inserted distally in the side channel to a depth at which the endoscope abuts the distal portion of the side channel. Unfortunately, however, although this approach may be beneficial in those cases where the surgeon wishes to position the endoscope at exactly the same depth that is defined by the distal portion of the side channel, this approach does not afford the surgeon any options for alternative depths to position the endoscope; consequently, this approach typically limits the surgeon to a single view at a defined depth, significantly constraining surgical flexibility and access. Moreover, as can be appreciated, this approach requires the use of a laryngoscope having a side channel of the type described above.

In addition to the aforementioned shortcomings, a shortcoming that is shared by both microscopic-guided laser surgery and endoscopic laser surgery is that the surgical laser is limited to accessing targets that are in-line with the exit opening of the surgical laser. Consequently, for targets that are not otherwise positioned in-line with the exit opening of the surgical laser, it may be necessary to use suction to draw the target in-line and/or to resect nearby tissue to draw the target in-line. As can readily be appreciated, however, such approaches create their own sets of risk.

Moreover, in cases in which the surgical laser is coupled to a handpiece held by the surgeon, laser targeting can be more susceptible to tremor, resulting in inaccuracies in the tissue being irradiated.

### SUMMARY OF THE INVENTION

It is an object of the present invention to provide one or more novel devices suitable for use in performing an otolaryngology procedure.

It is another object of the present invention to provide a novel device as described above that overcomes at least some of the shortcomings associated with existing devices suitable for use in performing an otolaryngology procedure.

Therefore, according to one aspect of the invention, there is provided an endoscope positioning device, the endoscope positioning device comprising (a) a frame, the frame being removably mountable on a proximal end of a laryngoscope blade; (b) a sheath, the sheath being disposed on the frame and being insertable over a portion of an endoscope; and (c) wherein the frame and the sheath comprise complementary means for coupling the sheath to the frame in a manner in which distal movement of the sheath relative to the frame is delimited.

In a more detailed feature of the invention, the sheath may comprise a jacket, the jacket may be insertable over a portion of an endoscope insertion tube, the frame may comprise a support, the sheath may be disposed on the support, and the complementary means may comprise a projection extending outwardly from the sheath and a first stop coupled to the support, wherein the first stop may be configured to engage the projection.

In a more detailed feature of the invention, the sheath may comprise a jacket, the jacket may be insertable over a portion of an endoscope insertion tube, the frame may comprise a support, the sheath may be disposed on the support, and the complementary means may comprise a projection extending outwardly from the sheath and first and second stops coupled to the support, wherein the first and second stops may be coupled to the support at different points along a length of the support, and wherein one of the first and second stops may be configured to engage the projection.

In a more detailed feature of the invention, the frame may comprise a guide, the guide may be disposed on the support and aligned therewith, and the guide may receive a portion of the sheath.

In a more detailed feature of the invention, the frame may comprise a clip, and the clip may be configured to be detachably secured to the proximal end of a laryngoscope blade.

In a more detailed feature of the invention, the sheath may be dimensioned to delimit axial movement of the endoscope relative to the sheath.

According to another aspect of the invention, there is provided an endoscope assembly, the endoscope assembly comprising the above-described endoscope positioning device and an endoscope, wherein the sheath of the endoscope positioning device may be removably inserted over a portion of the endoscope.

According to yet another aspect of the invention, there is provided a system suitable for use in performing an otolaryngology procedure, the system comprising a laryngoscope and the above-described endoscope assembly, wherein the laryngoscope may comprise a blade having a channel, wherein the endoscope assembly may be removably mounted on the blade of the laryngoscope, and wherein the endoscope may be inserted into the channel.

In a more detailed feature of the invention, the system may further comprise a laser beam targeting assembly, and the laser beam targeting assembly may be removably mounted on the blade of the laryngoscope.

In a more detailed feature of the invention, the laser beam targeting assembly may comprise a platform, the platform may be disposed in the channel, the platform may comprise a first docking port, and the first docking port may be configured to receive a surgical laser handpiece in an orientation to direct a laser beam emitted from the surgical laser handpiece distally through the channel.

In a more detailed feature of the invention, the platform may further comprise a second docking port, the laser beam targeting assembly may further comprise an angularly deflectable mirror coupled to the second docking port, and the angularly deflectable mirror may be configured to reflect the laser beam emitted from the surgical laser.

According to still yet another aspect of the invention, there is provided a kit suitable for use in performing an otolaryngology procedure, the kit comprising (a) a first frame, the first frame being removably mountable on a proximal end of a laryngoscope blade; (b) a sheath, the sheath being positionable on the first frame and being insertable over a portion of an endoscope; and (c) wherein the first frame and the sheath comprise first complementary means for coupling the sheath to the first frame in a manner in which distal movement of the sheath relative to the first frame is delimited.

In a more detailed feature of the invention, the sheath may comprise a jacket, the jacket may be insertable over a portion of an endoscope insertion tube, the first frame may comprise a first support, the sheath may be positionable on the first support, and the first complementary means may comprise a projection extending outwardly from the sheath and a first stop coupled to the first support, wherein the first stop may be engageable with the projection.

In a more detailed feature of the invention, the sheath may comprise a jacket, the jacket may be insertable over a portion of an endoscope insertion tube, the first frame may comprise a first support, the sheath may be positionable on the first support, wherein the first complementary means may comprise a projection extending outwardly from the sheath and first and second stops coupled to the first support, wherein the first and second stops may be positioned at different points along a length of the first support, and wherein the first and second stops may be alternatively engageable with the projection.

In a more detailed feature of the invention, each of the first and second stops may be transformable between a deployed state engageable with the projection and a non-deployed state not engageable with the projection.

In a more detailed feature of the invention, each of the first and second stops may be transformable by being bent manually.

In a more detailed feature of the invention, the kit may further comprise a second frame, the second frame may be removably mountable on the proximal end of the laryngoscope blade, the second frame and the sheath may comprise second complementary means for coupling the sheath to the second frame in a manner in which distal movement of the sheath relative to the second frame may be delimited, and the first frame and the second frame may be configured to position a distal end of an endoscope at different depths in the laryngoscope blade.

In a more detailed feature of the invention, the kit may further comprise a laser beam targeting assembly, the laser beam targeting assembly may comprise a platform removably mountable in a laryngoscope blade, the platform may comprise a first docking port, and the first docking port may be configured to receive a surgical laser handpiece in an orientation to direct a laser beam emitted from the surgical laser handpiece distally through the channel.

In a more detailed feature of the invention, the kit may further comprise a second docking port, the laser beam targeting assembly may further comprise an angularly deflectable mirror coupled to the second docking port, and the angularly deflectable mirror may be configured to reflect the laser beam emitted from the surgical laser.

In a more detailed feature of the invention, the laser beam targeting assembly may further comprise a ball mount and a wand, and the ball mount and the wand may couple the angularly deflectable mirror to the second docking port.

For purposes of the present specification and claims, various relational terms like “top,” “bottom,” “proximal,” “distal,” “upper,” “lower,” “front,” and “rear” may be used to describe the present invention when said invention is positioned in or viewed from a given orientation. It is to be understood that, by altering the orientation of the invention, certain relational terms may need to be adjusted accordingly.

Additional objects, as well as features and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. These drawings are not necessarily drawn to scale, and certain components may have undersized and/or oversized dimensions or may be shown in a simplified form for purposes of explication. In the drawings wherein like reference numerals represent like parts:

FIG. 1 is a front perspective view of one embodiment of a system suitable for use in performing an otolaryngology procedure, the system being constructed according to the teachings of the present invention;

FIG. 2 is a fragmentary bottom perspective view of the system of FIG. 1;

FIG. 3 is a side view of the laryngoscope shown in FIG. 1;

FIG. 4 is an enlarged fragmentary front view of the laryngoscope shown in FIG. 3;

FIG. 5 is a perspective view of the endoscope assembly shown in FIG. 1;

FIG. 6 is a partly exploded perspective view of the endoscope assembly shown in FIG. 5;

FIG. 7 is a perspective view of the endoscope shown in FIG. 5;

FIG. 8 is a top view of the endoscope sheath shown in FIG. 5;

FIG. 9 is a perspective view of the endoscope sheath shown in FIG. 8;

FIG. 10 is an enlarged fragmentary longitudinal section view of the endoscope sheath shown in FIG. 8;

FIG. 11 is a top view of the frame shown in FIG. 5;

FIG. 12 is a perspective view of the frame shown in FIG. 11;

FIG. 13 is an enlarged fragmentary side view of the frame shown in FIG. 11;

FIG. 14 is a top view of a first embodiment of an endoscope positioning kit constructed according to the present invention;

FIG. 15 is a top view of a second embodiment of an endoscope positioning kit constructed according to the present invention;

FIG. 16 is a perspective view of one embodiment of the laser beam targeting assembly shown in FIG. 1;

FIG. 17 is an enlarged fragmentary side view of the laser beam targeting assembly shown in FIG. 16;

FIG. 18 is an enlarged fragmentary longitudinal section view of the laser beam targeting assembly shown in FIG. 16;

FIG. 19 is a front view of the frame of the laser beam targeting assembly shown in FIG. 16;

FIG. 20 is a longitudinal section view of the frame of the laser beam targeting assembly shown in FIG. 16; and

FIG. 21 is an enlarged fragmentary side view of the wand shown in FIG. 16.

### DETAILED DESCRIPTION OF THE INVENTION

As noted above, existing approaches for performing otolaryngology procedures, particularly those otolaryngology procedures involving the use of an endoscope and/or a surgical laser, possess certain shortcomings. Accordingly, the present invention is directed at an approach that is suitable for use in performing such otolaryngology procedures and that minimizes at least some of the shortcomings that are associated with these existing approaches.

More specifically, whereas existing approaches typically involve either (i) requiring a surgeon to continuously hold an endoscope at a desired position (i.e., depth) in a channel of a laryngoscope blade or (ii) positioning the endoscope at a fixed depth defined by a narrowed distal portion of a side channel of a laryngoscope blade, one feature of the present invention is the provision of the capability to securely position an endoscope within a laryngoscope blade in a hands-free manner without the need for a side channel having a narrowed distal portion. Consequently, in at least some cases, the present invention may enable an endoscope to be securely positioned in a hands-free manner at a specifically desired depth or at different alternative depths.

Additionally, whereas existing approaches enable a surgical laser only to access targets that are in-line with the exit opening of the surgical laser, one feature of the present invention is the provision of the capability for the surgical laser not only to access targets that are in-line with the exit opening of the surgical laser but, alternatively, to access targets that are not in-line with the exit opening of the surgical laser. In this manner, techniques that are commonly used to alter the position of targets that are not otherwise in-line with the exit opening of the surgical laser to draw such targets in-line need not be employed.

To this end, as discussed further below, the present invention is directed, at least in part, at one or more first devices that may embody or possess the first feature described above, at one or more second devices that may embody or possess the second feature described above, at one or more kits that may be used to form the first device and/or the second device, at one or more systems that may comprise the one or more first devices and/or the one or more second devices, and at one or more methods of making and/or using the aforementioned devices, kits and/or systems.

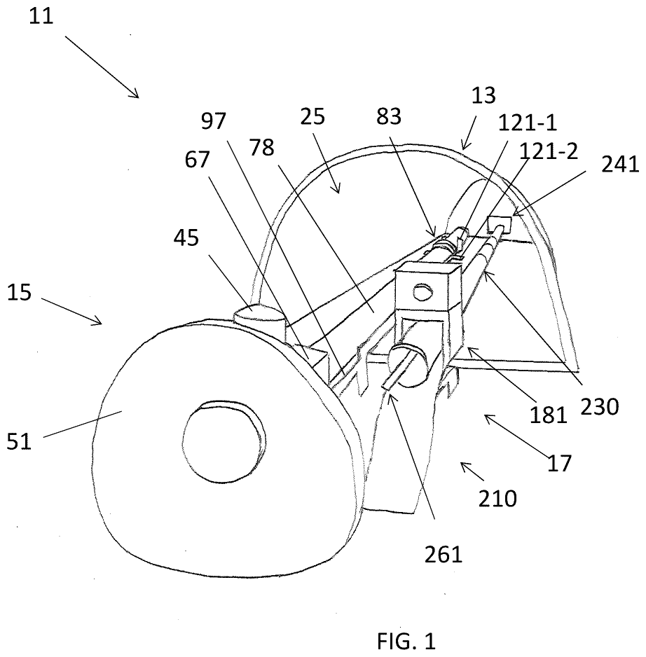

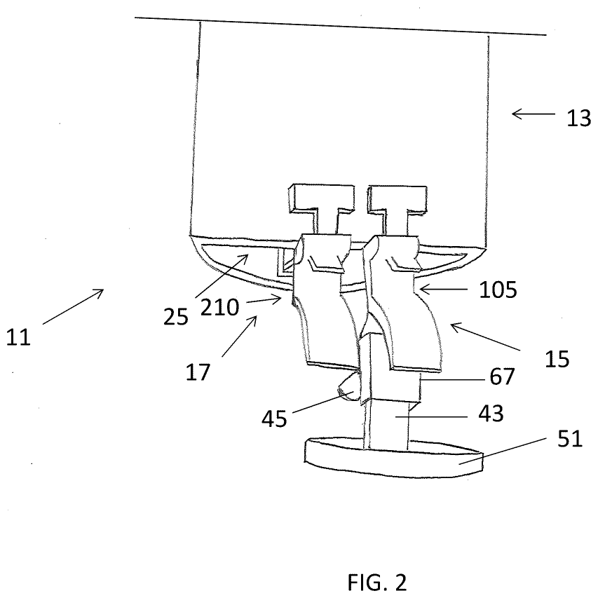

Referring now to FIGS. 1 and 2, there are shown various views of one embodiment of a system suitable for use in performing an otolaryngology procedure, the system being constructed according to the present invention and being represented generally by reference numeral 11. For clarity, simplicity, and/or ease of illustration, certain details or features of system 11 that are discussed elsewhere in this application or that are not critical to an understanding of the invention may be omitted from the present specification and/or may be omitted from one or more of FIGS. 1 and 2 or may be shown in one or more of FIGS. 1 and 2 in a simplified manner. For example, in FIGS. 1 and 2, the handle of the laryngoscope is not shown.

System 11 may comprise a laryngoscope 13, an endoscope assembly 15, and a laser beam targeting assembly 17.

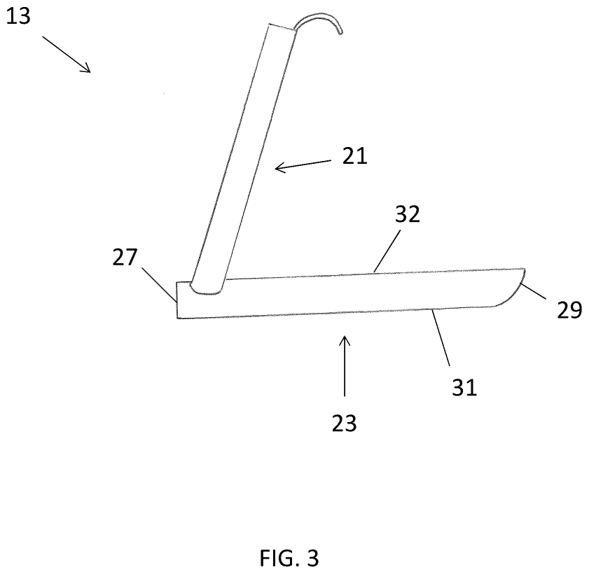

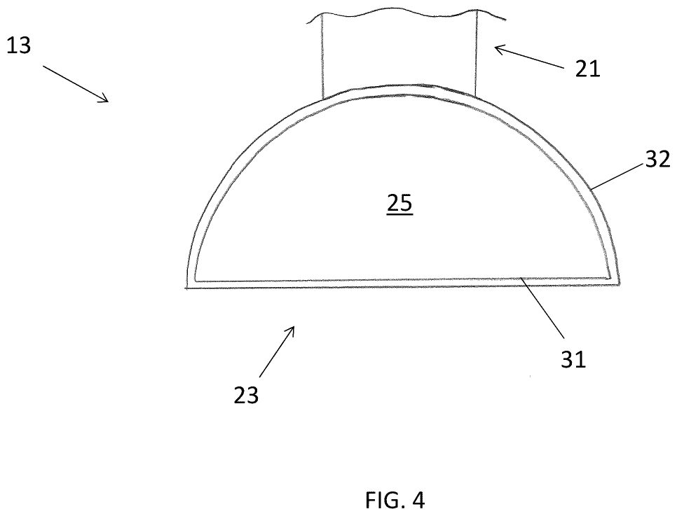

Laryngoscope 13, which is also shown separately in FIGS. 3 and 4, may be a conventional laryngoscope of the type commonly used by otolaryngologists in otolaryngology procedures. As such, laryngoscope 13 may comprise a handle 21 and a blade 23, wherein handle 21 may extend generally upwardly from blade 23 at nearly a right angle. In the present embodiment, blade 23 may comprise a rigid structure having a channel 25 that extends longitudinally from a proximal end 27 of blade 23 to a distal end 29 of blade 23. In the present embodiment, channel 25 may be generally semi-cylindrical in shape and may have a generally flat bottom wall 31 and an arcuate top wall 32. Notwithstanding the above, it is to be understood that channel 25 need not be semi-cylindrical in shape and/or need not have a flat bottom wall 31 or an arcuate top wall 32. Moreover, although blade 23 of the present embodiment has only a single channel 25, blade 23 could be constructed to include a plurality of generally longitudinally-extending channels including, but not limited to, a main channel and a side channel, wherein the side channel may have a narrowed distal portion to delimit the depth to which an endoscope may be inserted thereinto.

Laryngoscope 13 is preferably a multi-use item that is sterilized between uses.

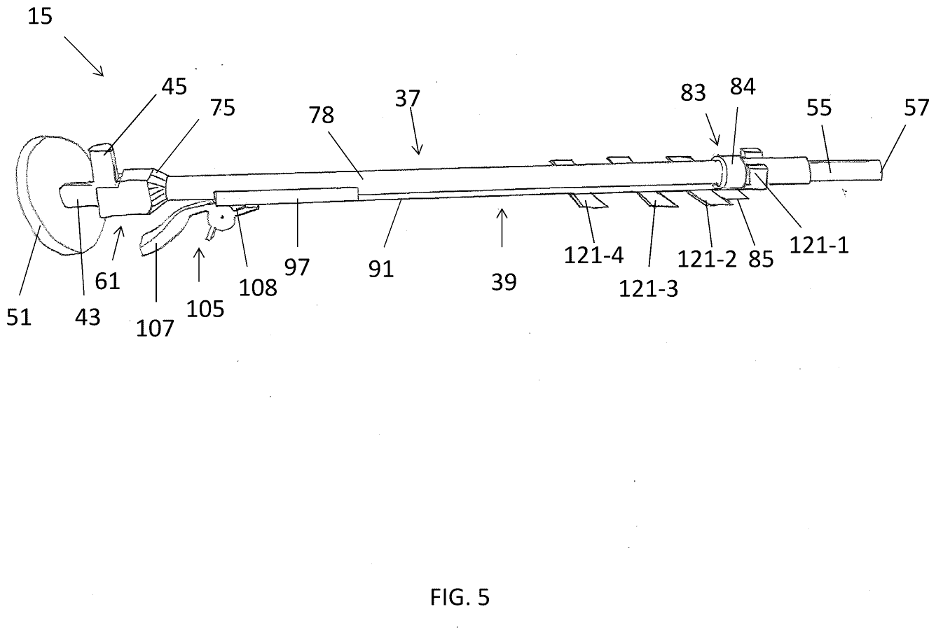

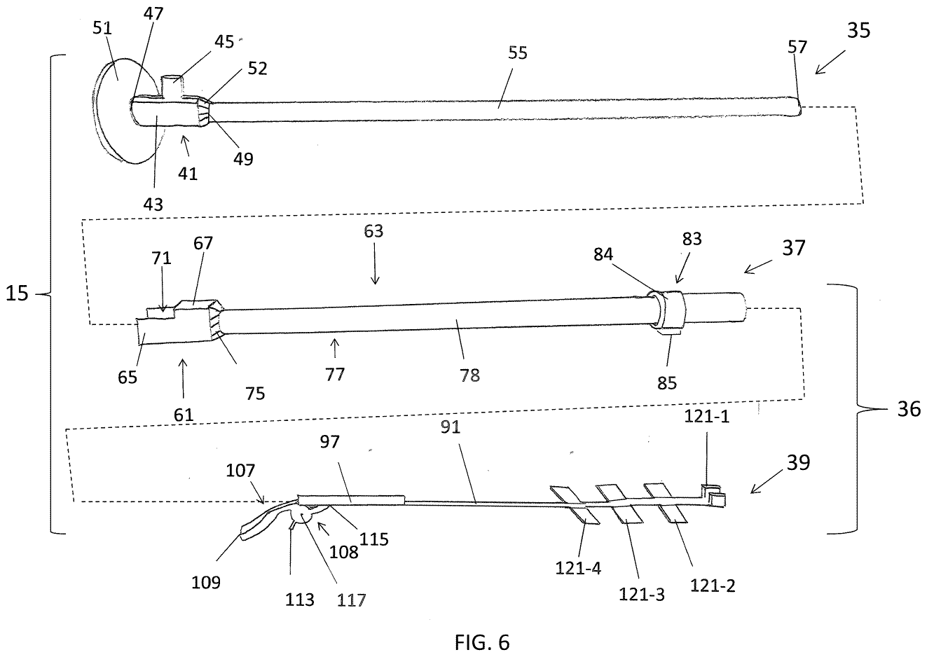

Endoscope assembly 15, which is also shown separately in FIGS. 5 and 6, may comprise an endoscope 35 and an endoscope positioning device 36.

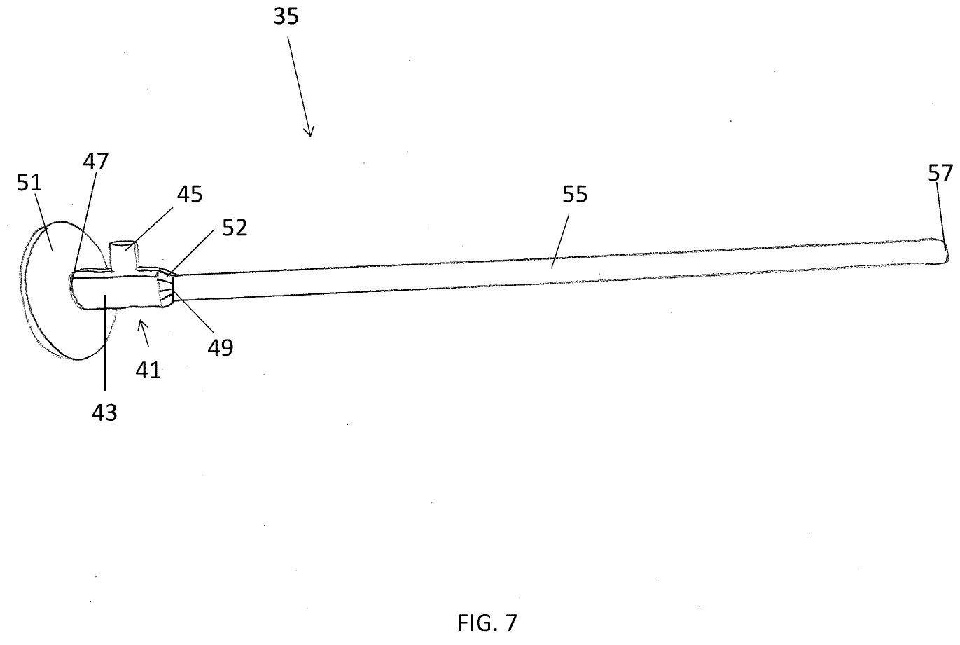

Endoscope 35, which is also shown separately in FIG. 7, may be identical to a conventional endoscope. Accordingly, endoscope 35 may comprise a handpiece 41. Handpiece 41, in turn, may comprise a main portion 43 and a side portion 45. Main portion 43 may include a tubular structure extending longitudinally between a proximal end 47 and distal end 49. An eyepiece 51 may be operatively coupled to proximal end 47 of handpiece 41 for viewing an image of an illuminated target. (If desired eyepiece 51 may be coupled to an external monitor (not shown) so that the image of the illuminated target may be displayed on the external monitor.) A distal portion 52 of main portion 43 terminating at distal end 49 may be frustoconical in shape, tapering distally.

Side portion 45 of handpiece 41 may be arranged generally perpendicular to main portion 43 of handpiece 41 and may be positioned at a point that is intermediate to proximal end 47 and distal end 49. Side portion 45 may be configured to be optically coupled to a light source (not shown) to provide illuminating light to endoscope 35.

Endoscope 35 may further comprise an insertion tube 55. Insertion tube 55 may comprise an elongated tubular structure having a generally uniform outer diameter over its length. Insertion tube 55 may extend distally from handpiece 41 and may terminate at a distal end 57. Although not shown, insertion tube 55 may house one or more optical fibers optically coupled to side portion 45 for use in illuminating a target, as well as housing an objective lens and one or more optical fibers for use in imaging the illuminated target. In the present embodiment, insertion tube 55 is rigid; however, insertion tube 55 need not be rigid and, if desired, may be flexible.

Endoscope 35 may be optically configured to image a target that is positioned along the longitudinal axis of insertion tube 55 (i.e., a 0-degree endoscope). Alternatively, endoscope 35 may be optically configured to image a target that is positioned at a 90-degree angle relative to the longitudinal axis of insertion tube 55 (i.e., a 90-degree endoscope) or may be optically configured to image a target at some intermediate angle, such as, for example, 30 degrees, 45 degrees, or 70 degrees.

Endoscope 35 is preferably a multi-use item that is sterilized between uses.

Endoscope positioning device 36, which may be used to securely, but removably, position endoscope 35 within channel 25 of laryngoscope blade 23, may comprise a sheath 37 and a frame 39.

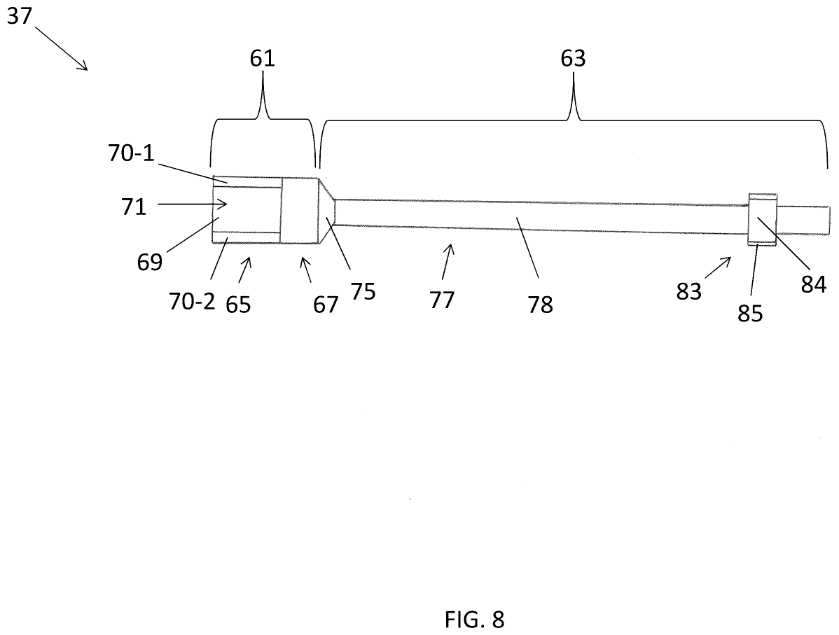

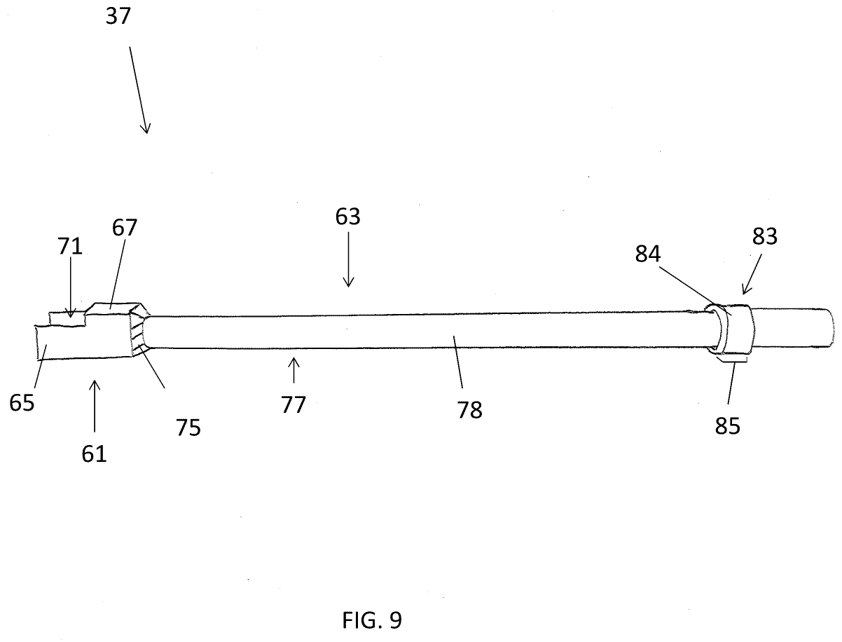

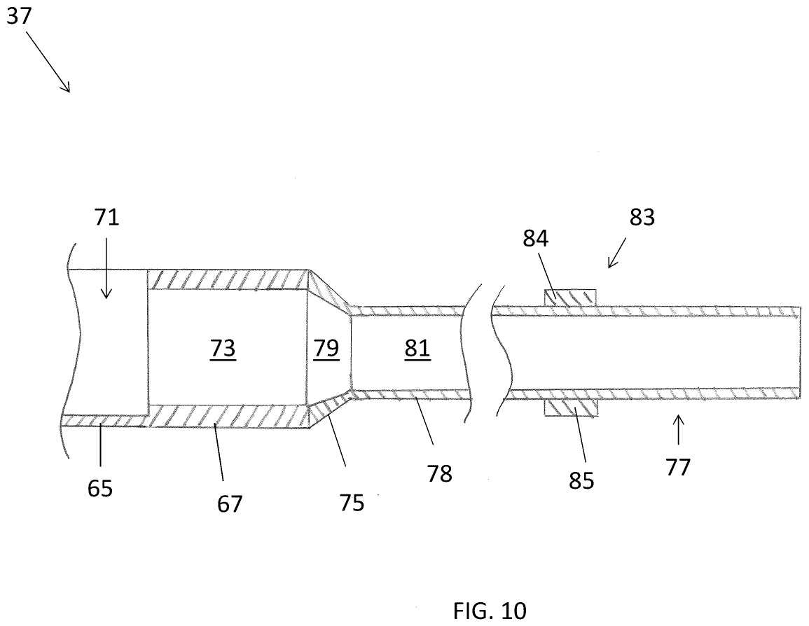

Sheath 37, which is also shown separately in FIGS. 8, 9, and 10, may comprise a proximal portion 61 and a distal portion 63. In the present embodiment, proximal portion 61 and distal portion 63 may be formed as a unitary (i.e., one-piece) structure; however, this need not be the case as proximal portion 61 and distal portion 63, or portions thereof, may be constructed separately and then joined together. Proximal portion 61 may consist of or comprise a rigid, yet lightweight, material, such as a suitable polymeric or metallic material, and may be molded or otherwise constructed to include a front section 65 and a rear section 67.

Front section 65 may be shaped to include a bottom wall 69 and two side walls 70-1 and 70-2, which collectively may define a slot 71 having an open front and an open top. Slot 71 may be appropriately dimensioned to receive a first portion of main portion 43 of endoscope 35, with side portion 45 of endoscope 35 extending through the open top of slot 71. Rear section 67 may comprise a generally rectangular prismatic block, which may be shaped to include a longitudinal bore 73. Longitudinal bore 73 may be aligned with slot 71 and may be appropriately dimensioned to receive a second portion of main portion 43 of handpiece 41.

Distal portion 63 of sheath 37 may consist of or comprise a rigid, yet lightweight, material, such as a suitable polymeric or metallic material, and may be molded or otherwise constructed to include a front section 75 and a rear section 77. Front section 75 may be frustoconical in shape and may have a tapered bore 79 of corresponding shape, with the wider end of bore 79 being aligned with longitudinal bore 73 of rear section 67. Preferably, bore 79 is appropriately dimensioned to mate with distal portion 52 of handpiece 41 in a friction-fit, thereby enabling endoscope 35 to be removably secured to sheath 37 and delimiting distal movement of endoscope 35 relative to sheath 37. Rear section 77 may comprise an elongated jacket 78 of generally cylindrical shape having a longitudinal bore 81 of corresponding shape. Bore 81 may be in communication with bore 79 and may be appropriately dimensioned in cross-section to receive insertion tube 55 of endoscope 35. Jacket 78 may be dimensioned to have a shorter length than that of insertion tube 55 of endoscope 35 so that jacket 78 does not interfere with visualization at the viewing end of insertion tube 55.

Rear section 77 of sheath 37 may also be shaped to include a projection 83 extending radially outwardly from the exterior surface of jacket 78. In the present embodiment, projection 83 may be in the form of a collar or bumper circumferentially surrounding jacket 78, said collar including an arcuate top portion 84 and a flat bottom portion 85. The purpose of projection 83 will be discussed below; however, it is to be understood that projection 83 need not be of the aforementioned shape and need not circumferentially surround jacket 78. Also, although, in the present embodiment, projection 83 is formed as part of rear section 77 of sheath 37 and, thus, is fixed at a particular axial position along the length of jacket 78, one could construct projection 83 separately from jacket 78 and, thus, secure projection 83 to jacket 78 at a different axial position than that shown or could construct projection 83 so as to be adjustably secured to jacket 78 at any of a plurality of alternative axial positions. In the present embodiment, projection 83 has a static size; however, it is to be understood that, in another embodiment (not shown), projection 83 could be deployable or transformable from a smaller size to a larger size to facilitate engagement with frame 39 in the manner to be discussed below.

In the present embodiment, sheath 37 may be separated from endoscope 35 after a single use, and then sheath 37 may be disposed of or recycled in an appropriate fashion. That being said, alternatively, sheath 37 may be used as a multi-use item that is sterilized between uses; thus, in an alternative embodiment, sheath 37 may be removed from endoscope 35 after use, sterilized, and then remounted on the same or different endoscope.

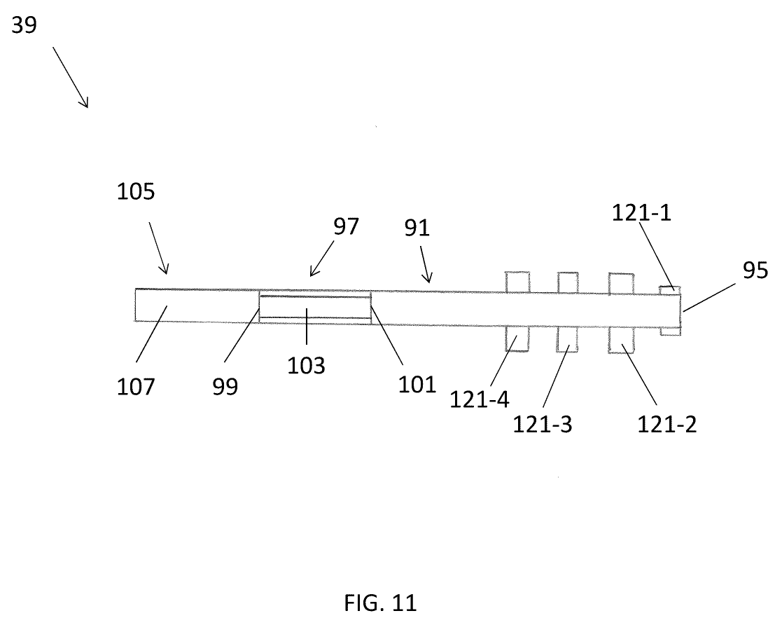

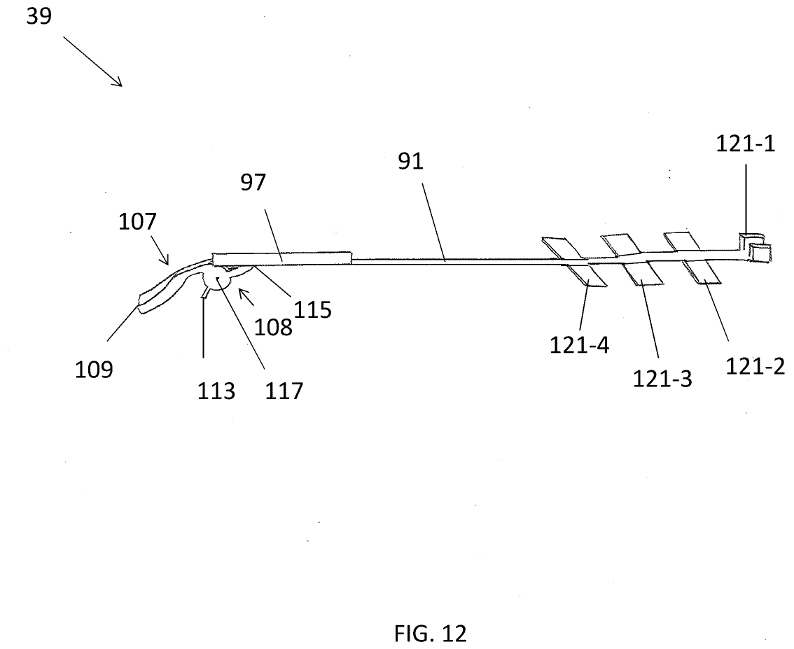

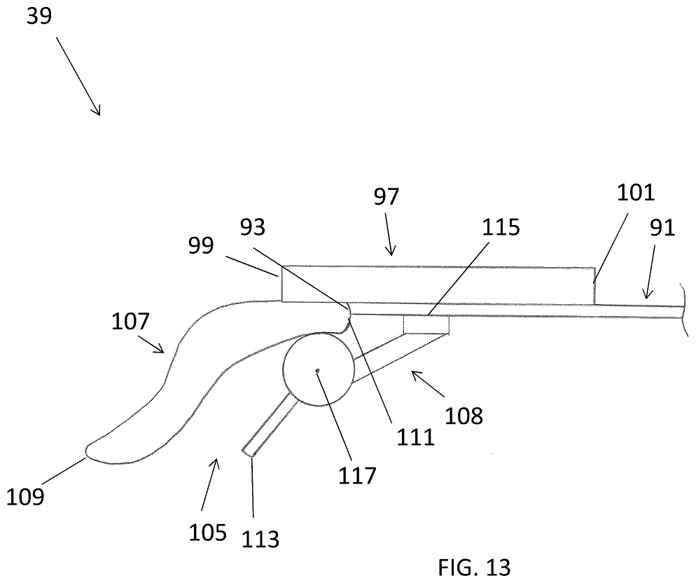

Frame 39, which is also shown separately in FIGS. 11 through 13, may comprise a support 91. In the present embodiment, support 91 may consist of or comprise a rigid, yet lightweight, material, such as a suitable polymeric or metallic material. Support 91 may be substantially flat or planar and may be constructed to comprise an elongated rectangular member having a proximal end 93 and a distal end 95. Support 91 may be dimensioned to have a length such that support 91 does not extend beyond distal end 29 of blade 23. The dimensions of support 91 may correspond to specific laryngoscope sizes, with variations accommodating both pediatric and adult instruments. The width of support 91 may closely match the diameter of sheath 37 to maintain proper alignment throughout the system.

Frame 39 may further comprise a guide 97. In the present embodiment, guide 97 may be similar in composition to support 91 and may comprise an elongated trough-shaped member having an open proximal end 99, an open distal end 101, and an arcuate channel 103. Guide 97 may be dimensioned to have a width similar to that of support 91 but a length considerably shorter than that of support 91. Guide 97 may be fixedly mounted on top of support 91. In the present embodiment, the open proximal end 99 of guide 97 may be positioned proximally a short distance relative to proximal end 93 of support 91. Arcuate channel 103 may be appropriately dimensioned to slidably receive rear section 77 of sheath 37. In this manner, guide 97 may be used to guide the alignment of rear section 77 of sheath 37 relative to support 91 while preventing side-to-side motion of sheath 37 relative to support 91, thereby enabling frame 39 to delimit distal movement of sheath 37 in the manner to be discussed below.

It is to be understood that, although, in the present embodiment, support 91 and guide 97 are described as being separate components, one could make support 91 and guide 97 as a unitary structure.

Frame 39 may further comprise a clip 105. In the present embodiment, clip 105 may comprise a stationary leg 107 and a movable leg 108. Stationary leg 107 may have a proximal end 109 and a distal end 111. Proximal end 109 may be constructed to enable the manual manipulation thereof, for example, using the thumb of an operator. Distal end 111 of stationary leg 107 may be fixedly mounted on proximal end 93 of support 91. In addition, stationary leg 107 may be fixedly mounted at a point proximate to distal end 111 on a bottom surface of guide 97.

Movable leg 108 may have a proximal end 113 and a distal end 115. Proximal end 113 of movable leg 108 may be constructed to enable the manual manipulation thereof, for example, using the forefinger of an operator. Stationary leg 107 and movable leg 108 may be coupled to one another about a pivot 117, and biasing means, such as a spring (not shown), may be used to bias distal end 115 of movable leg 108 upwardly in the direction of support 91. As a result, frame 39 may be securely, but removably, mounted on the proximal end of bottom wall 31 of laryngoscope blade 23, for example, by using the thumb and the forefinger of an operator to pivot proximal end 113 of movable leg 108 upwardly towards proximal end 109 of stationary leg 107, thereby causing distal end 115 of movable leg 108 to be rotated downwardly; then, with movable leg 108 still rotated downwardly, by inserting the proximal end of bottom wall 31 of laryngoscope blade 23 between stationary leg 107 and movable leg 108; and, then, by releasing proximal end of 113 of movable leg 108, thereby causing distal end 115 of movable leg 108 to engage the exterior bottom surface of bottom wall 31 of laryngoscope blade 23. Correspondingly, to decouple frame 39 from laryngoscope blade 23, one may pivot proximal end 113 of movable leg 108 upwardly towards proximal end 109 of stationary leg 107, thereby causing distal end 115 of movable leg 108 to be rotated downwardly; and, then, with movable leg 108 still rotated downwardly, one may remove the proximal end of laryngoscope blade 23 from between stationary leg 107 and movable leg 108. Thereafter, proximal end of 113 of movable leg 108 may be released, thereby closing clip 105.

It is to be understood that, although, in the present embodiment, clip 105 is disclosed as being secured to bottom wall 31 of laryngoscope blade 23, clip 105 is not limited to securement to bottom wall 31 of laryngoscope blade 23 and may be secured, for example, to arcuate top wall 32 of laryngoscope blade 23.

Frame 39 may further comprise a plurality of stops 121-1 through 121-4, which may be alternatively deployed in the manner discussed below to engage projection 83. (In the present embodiment, stop 121-1 is shown in a deployed state, and stops 121-2, 121-3 and 121-4 are shown in a non-deployed state; however, it is to be understood that, alternatively, stop 121-2 or stop 121-3 or stop 121-4 could be deployed, and the other three stops may be non-deployed.) In the present embodiment, each of stops 121-1 through 121-4 may comprise a strip or band of a bendable, yet sturdy, material capable of being stably transformed, by hand, from a non-deployed or generally planar state to a deployed or deflected state. More specifically, in the present embodiment, each of stops 121-1 through 121-4 may be fixedly secured to the bottom surface of support 91 at different points along the length of support 91. Stops 121-1 through 121-4 may be appropriately dimensioned and oriented relative to support 91 so as to have a pair of deflectable tabs extending past support 91 on opposite sides thereof. When not deployed, the tabs of stops 121-1 through 121-4 may lie flat. By contrast, when deployed, the tabs of a given stop may be deflected upwardly at a desired angle to an extent sufficient to prevent projection 83 on sheath 37 from moving distally past the deflected stop. Accordingly, in this manner, by selectively deploying a different one of stops 121-1 through 121-4, one may select the extent to which sheath 37 may be moved distally relative to frame 39 and, in so doing, may select the depth, relative to laryngoscope blade 23, to which endoscope 35 may be inserted. The selective deployment of stops 121-1 through 121-4 leg position may be achieved in various ways. For example, according to one way, a stop may be deployed using a screw-based fixation system, whereby the stop is bent and then is held in place by a screw prior to insertion of frame 39 in blade 23. According to an alternative way, a spring-loaded mechanism, which may be activated remotely by a proximal control located outside the patient and while the frame is mounted within blade 23, may be employed. Such a remote mechanism may cause deployment (flip up from the horizontal to vertical position) through a button-type actuation at the surgeon's end. Regardless of the means used to deploy a stop, only one stop should be deployed at any given time for safety and operational stability.

It is to be understood that, although, in the present embodiment, stops 121-1 through 121-4 are disclosed as having a particular shape, this need not be the case as stops 121-1 through 121-4 merely need to be designed so that, when a given stop is deployed, the deployed stop engages projection 83 sufficiently to delimit axial movement of sheath 37 relative to frame 39. Thus, each of projection 83 and stops 121-1 through 121-4 may assume a myriad of shapes.

It is also to be understood that, although, in the present embodiment, stops 121-1 through 121-4 are described as being fixed to support 91, one could mount stops 121-1 through 121-4 on support 91 in such a manner that the axial positioning of one or more of stops 121-1 through 121-4 relative to support 91 may be adjusted and then re-secured one or more times, thereby enabling endoscope 35 to be positionable at additional depths from those shown. For example, one or more of stops 121-1 through 121-4 may be capable of being releasably secured to support 91 along either a continuum of points along the length of support 91 or at one or more spaced-apart intervals along support 91, for example, by tightening/loosening a screw used to couple together the stop and support 91.

Although not shown, the deflected tabs of a deployed stop may be held in place using, for example, screws inserted through the deflected tabs and into support 91; alternatively, a retaining clip, a retaining bracket, or other structure may be used to maintain the tabs of a deployed stop in a deflected state.

It is to be understood that, although, in the present embodiment, frame 39 is shown as having four stops 121-1 through 121-4, frame 39 is not limited to having four stops and may have more than four stops or as few as one stop.

Additionally, it is also to be understood that, although, in the present embodiment, stops 121-1 through 121-4 are shown as being constructed as separate components from one another and from support 91, one could make stops 121-1 through 121-4 and support 91 (and guide 93) as a unitary structure.

Moreover, it is also to be understood that, although, in the present embodiment, stops 121-1 through 121-4 are described as being deployed by the manual deflection of tabs, one could deflect the tabs of a desired stop by means other than manual deflection. For example, the deployment of stops 121-1 through 121-4 may be achieved using electromechanical means (e.g., one or more motors), which may be controlled remotely via wireless or wired connections. Alternatively, the deployment of stops 121-1 through 121-4 may be achieved using hydraulic or pneumatic means, such as strategically placed bladders that may be independently filled and/or emptied with water or air, using, for example, remotely-positioned and/or remotely-controlled syringes or the like. Alternatively, stops 121-1 through 121-4, themselves, may comprise independently deployable bladders that may be deployed using hydraulic or pneumatic means. The selective transformation of stops 121-1 through 121-4 between deployed and non-deployed states to create different stopping points for sheath 36 may be actuated through proximal controls accessible to a surgeon or other medical personnel.

Furthermore, it is also to be understood that, although, in the present embodiment, the axial movement of sheath 37 relative to frame 39 is delimited by the interaction of projection 83 and stops 121-1 through 121-4, one could delimit the axial movement of sheath 37 relative to frame 39 by other means. For example, projection 83 and stops 121-1 through 121-4 could be replaced with various types of complementary fixing elements, such as hook-loop fasteners, snap fasteners, complementary threaded fasteners, and the like. Alternatively, sheath 37 and frame 39 may be releasably secured to one another adhesively, for example, by providing a suitable repositionable adhesive on one or both of sheath 37 and frame 39. Alternatively, sheath 37 and frame 39 may be releasably secured to one another magnetically, for example, by providing a permanent magnet on sheath 37 and an electromagnet on frame 39 that can be actuated remotely.

Frame 39 is preferably a multi-use item that is sterilized between uses.

To use endoscope positioning device 36 to securely, but removably, position endoscope 35 within channel 25 of laryngoscope blade 23, one may secure frame 39 to laryngoscope blade 23 using clip 105; one may insert endoscope 35 into sheath 37; one may position sheath 37 on frame 39; and one may deploy one of stops 121-1 through 121-4 on frame 39. The above steps may be performed sequentially in any order. Alternatively, two or more of these steps may be performed concurrently. After use, one may remove frame 39 from laryngoscope blade 23 using clip 105; one may remove endoscope 35 from sheath 37; and one may separate sheath 37 from frame 39. The above steps may be performed sequentially in any order. Alternatively, two or more of these steps may be performed concurrently.

It is to be understood that, although, in the present embodiment, endoscope positioning device 36 is disclosed as being used to position an endoscope within laryngoscope blade 23 having a single channel, endoscope positioning device 36 could alternatively be used to position an endoscope within either a first channel (e.g., a main channel) or a second channel (e.g., a side channel) of a laryngoscope blade having multiple channels. Where, for example, endoscope positioning device 36 is used to position an endoscope in a laryngoscope side channel having a narrowed distal portion, endoscope positioning device 36 may be dimensioned to position the endoscope in the side channel at a depth that is proximal to the narrowed distal portion, thereby enabling the endoscope to be secured in the side channel at an alternative depth to that provided by the narrowed distal portion.

Also, it is to be understood that, although, in the present embodiment, endoscope positioning device 36 is shown being used to position an endoscope within laryngoscope blade 23, endoscope positioning device 36 is not limited to such a use and may alternatively be used to position an endoscope or another device of similar structure relative to another type of device.

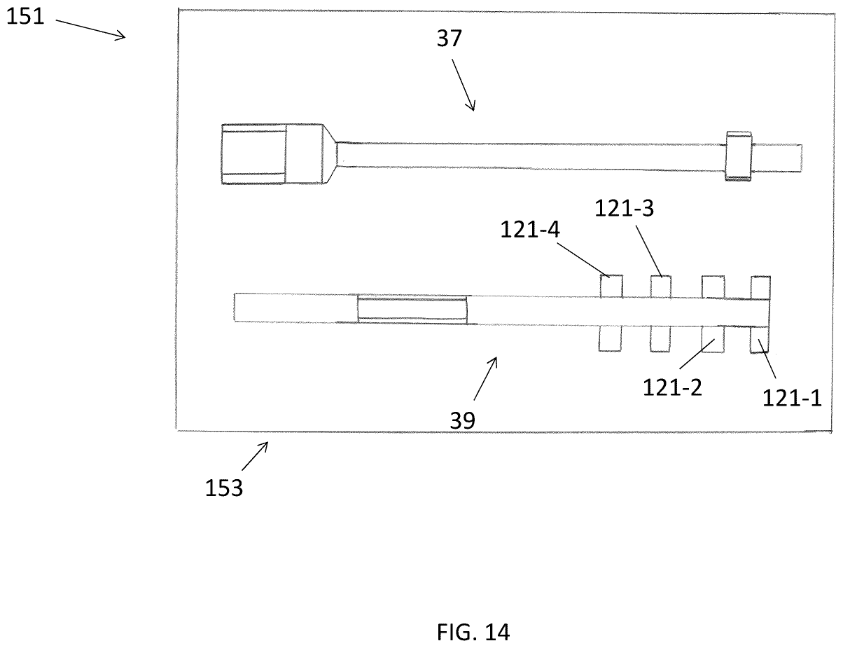

Sheath 37 and frame 39 may be provided together in an unassembled form as an endoscope positioning kit. Referring now to FIG. 14, there is shown a first embodiment of such an endoscope positioning kit according to the present invention, the endoscope positioning kit being represented generally by reference numeral 151. For clarity, simplicity, and/or ease of illustration, certain details or features of kit 151 that are discussed elsewhere in this application or that are not critical to an understanding of the invention may be omitted from the present specification and/or may be omitted from FIG. 14 or may be shown in FIG. 14 in a simplified manner.

As can be seen, endoscope positioning kit 151 may comprise sheath 37 and frame 39 in an unassembled state. Preferably, stops 121-1 through 121-4 of frame 39 are in a non-deployed state. Sheath 37 and frame 39 may be packaged in a sterile condition within sealed packaging 153. Although not shown, kit 151 may further comprise one or more additional sheaths, which may be identical to sheath 37, and/or may further comprise one or more additional frames, which may be identical to frame 39.

To use kit 151, packaging 153 may be opened, and sheath 37 and frame 39 may be used in the manner discussed above.

In another embodiment (not shown), kit 151 may further comprise laser beam targeting assembly 17 and/or laryngoscope 13, as well as other common accessories.

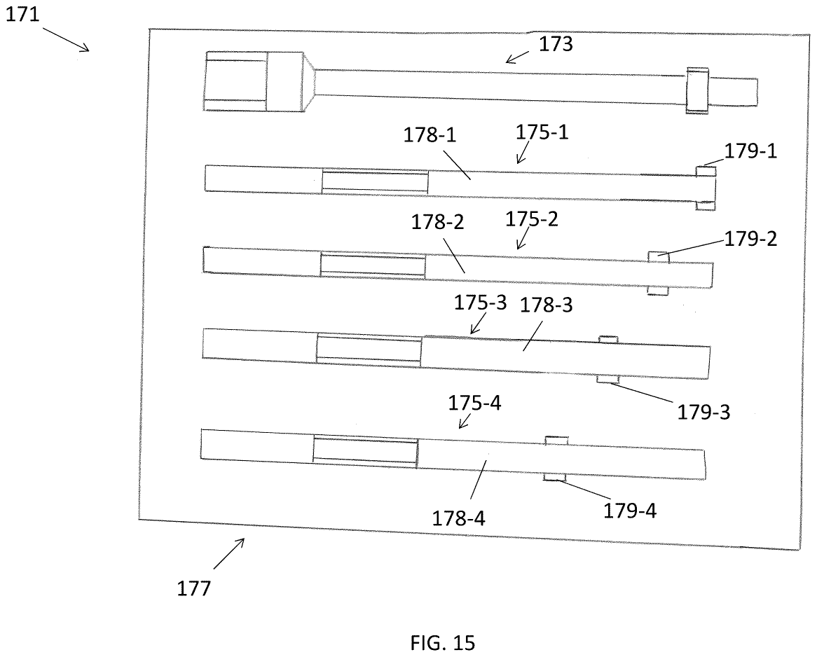

Referring now to FIG. 15, there is shown a second embodiment of an endoscope positioning kit according to the present invention, the endoscope positioning kit being represented generally by reference numeral 171. For clarity, simplicity, and/or ease of illustration, certain details or features of kit 171 that are discussed elsewhere in this application or that are not critical to an understanding of the invention may be omitted from the present specification and/or may be omitted from FIG. 15 or may be shown in FIG. 15 in a simplified manner.

Kit 171 may comprise a sheath 173 and a plurality of frames 175-1 through 175-4, all of which may be packaged in a sterile condition within sealed packaging 177. Sheath 173 may be identical to sheath 37. Frames 175-1 through 175-4 may be similar to frame 39; however, whereas frame 39 may comprise a plurality of stops 121-1 through 121-4 positioned at different points along the length of support 91, each of frames 175-1 through 175-4 may comprise a single stop, the various stops being positioned at different points along the length of their respective supports. In other words, frames 175-1 through 175-4 may comprise similarly dimensioned supports 178-1 through 178-4; however, frame 175-1 may comprise a single stop 179-1 positioned at a first distance from the distal end of support 178-1; frame 175-2 may comprise a single stop 179-2 positioned at a second distance (greater than the first distance) from the distal end of support 178-2; frame 175-3 may comprise a single stop 179-3 positioned at a third distance (greater than the second distance) from the distal end of support 178-3; and frame 175-4 may comprise a single stop 179-4 positioned at a fourth distance (greater than the third distance) from the distal end of support 178-4. In this manner, by selecting a desired frame to be used with sheath 173, one may control the depth to which an endoscope may be inserted. It should be noted that, in the present embodiment, each of stops 179-1 through 179-4 is shown in a deployed (i.e., bent) state; however, it is to be understood that stops 179-1 through 179-4 may be provided in kit 171 in a non-deployed state, with one or more of the stops, thereafter, being transformed into a deployed state.

In another embodiment (not shown), kit 171 may further comprise laser beam targeting assembly 17 disposed within packaging 177.

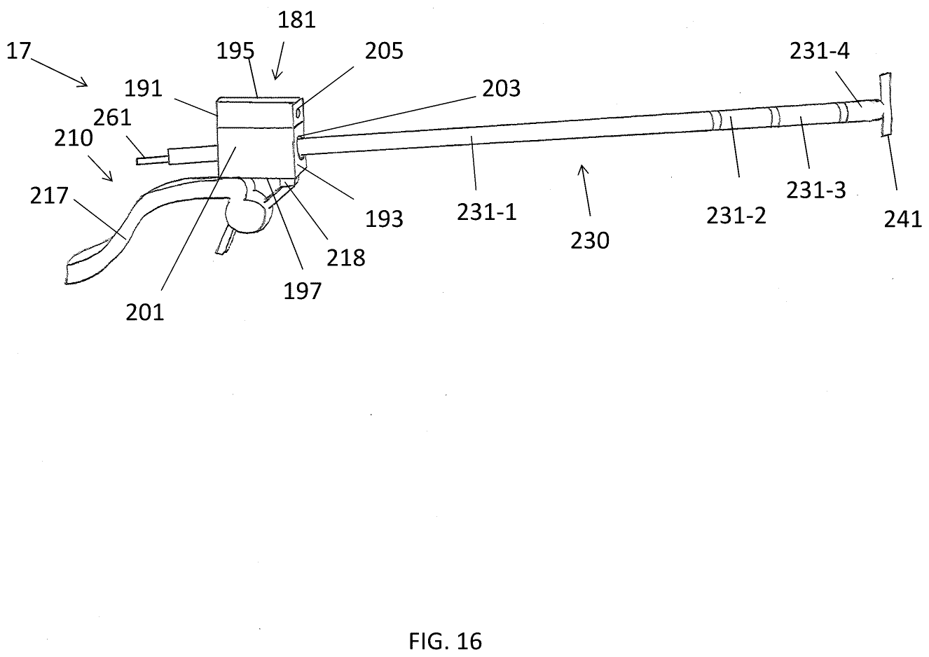

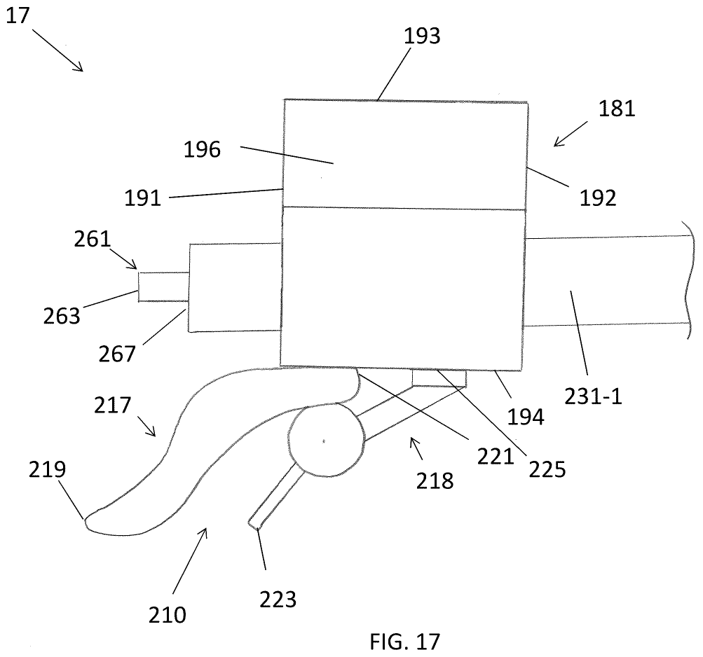

Referring back now to FIGS. 1 and 2 and referring additionally to FIGS. 16 through 18, laser beam targeting assembly 17 is shown in greater detail. For clarity, simplicity, and/or ease of illustration, certain details or features of laser beam targeting assembly 17 that are discussed elsewhere in this application or that are not critical to an understanding of the invention may be omitted from the present specification and/or may be omitted from one or more of FIGS. 16 through 18 or may be shown in one or more of FIGS. 16 through 18 in a simplified manner.

Laser beam targeting assembly 17 may comprise a frame or platform 181.

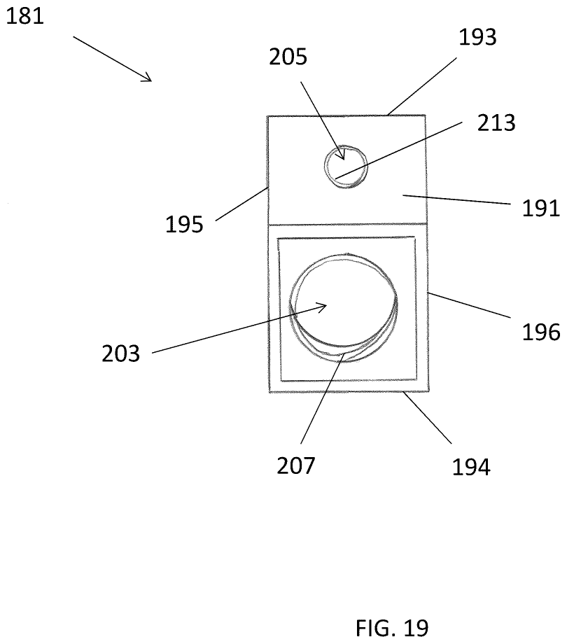

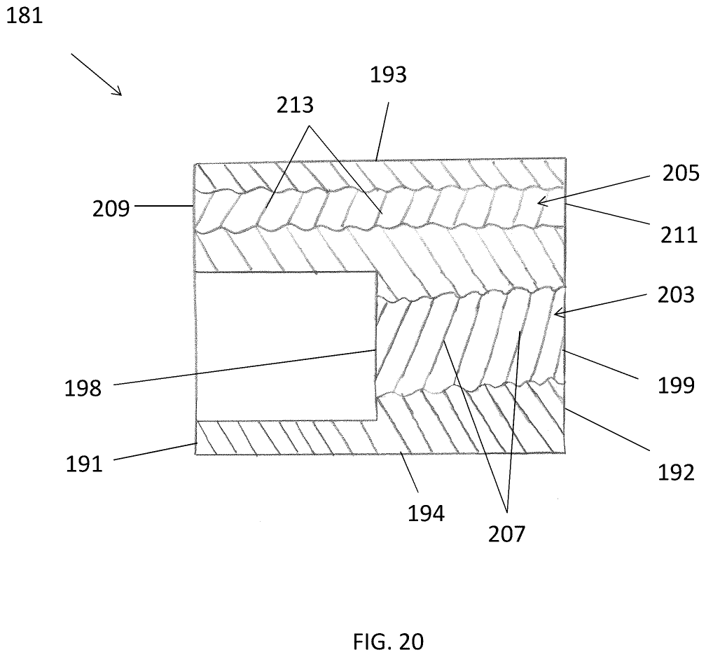

Frame 181, which is also shown separately in FIGS. 19 and 20, may comprise a block of generally rectangular prismatic shape having a front 191, a rear 192, a top 193, a bottom 194, a left side 195, and a right side 196. Frame 181 may consist of or comprise a rigid, yet lightweight, material, such as a suitable polymeric or metallic material. In the present embodiment, frame 181 may be a unitary structure; however, this need not be the case as portions thereof (such as a top portion and a bottom portion) may be constructed separately and then joined together.

Frame 181 may be shaped to include a first bore or docking port 203 and a second bore or docking port 205. First bore 203, which may be spaced upwardly a short distance from bottom 194 and which may be recessed a short distance from front 191, may be sloped slightly upwardly from its proximal end 198 to its distal end 199 for reasons to become apparent below. First bore 203 may be shaped to include an internal thread 207. As will be discussed further below, first bore 203 may be appropriately dimensioned to matingly receive a wand.

Second bore 205, which may be spaced upwardly a short distance from first bore 203, may be level (i.e., parallel to bottom 194 of frame 181) from its proximal end 209 to its distal end 211 for reasons to become apparent below. Second bore 205 may be appropriately dimensioned and shaped to include an internal thread 213 so that second bore 205 may be used to matingly receive an externally threaded handpiece (not shown) carrying a cable operatively coupled to the output of a surgical laser. Thus, second bore 205 may enable precise alignment of the laser beam from the surgical laser with the longitudinal axis of blade 23.

It is to be understood that, although, in the present embodiment, second bore 205 is positioned above first bore 203, one could position first bore 203 and second bore 205 in a side-by-side configuration, or one could position first bore 203 above second bore 205, or one could position first bore 203 and second bore 205 in still other configurations.

Laser beam targeting assembly 17 may further comprise a clip 210. Clip 210, which may be similar or identical to clip 105, may comprise a stationary leg 217 and a movable leg 218. Stationary leg 217 may have a proximal end 219 and a distal end 221, wherein distal end 221 may be fixedly mounted on bottom 194 of frame 181. Movable leg 218 may have a proximal end 223 and a distal end 225, wherein distal end 225 may be biased upwardly towards bottom 194 of frame 181. As a result, clip 210 (and frame 181, which is fixed to clip 210) may be securely, but removably, mounted on the proximal end of bottom wall 31 of laryngoscope blade 23, for example, by using the thumb and the forefinger of an operator to pivot proximal end 223 of movable leg 218 upwardly towards proximal end 219 of stationary leg 217, thereby causing distal end 225 of movable leg 218 to be rotated downwardly; then, with movable leg 218 still rotated downwardly, by inserting the proximal end of bottom wall 31 of laryngoscope blade 23 between stationary leg 217 and movable leg 218; and, then, by releasing proximal end of 223 of movable leg 218, thereby causing distal end 225 of movable leg 218 to engage the exterior bottom surface of bottom wall 31 of laryngoscope blade 23. Correspondingly, to remove clip 210 (and frame 181) from laryngoscope blade 23, one may pivot proximal end 223 of movable leg 218 upwardly towards proximal end 219 of stationary leg 217, thereby causing distal end 225 of movable leg 218 to be rotated downwardly; and, then, with movable leg 218 still rotated downwardly, one may remove the proximal end of laryngoscope blade 23 from between stationary leg 217 and movable leg 218. Thereafter, proximal end of 223 of movable leg 218 may be released, thereby closing clip 210.

It is to be understood that, although, in the present embodiment, clip 210 is disclosed as being secured to bottom wall 31 of laryngoscope blade 23, clip 210 is not limited to securement to bottom wall 31 of laryngoscope blade 23 and may be secured, for example, to arcuate top wall 32 of laryngoscope blade 23. Moreover, it is to be understood that, although, in the present embodiment, endoscope positioning device 36 and laser beam targeting assembly 17 are disclosed as being secured to the same wall of laryngoscope blade 23, this need not be the case as endoscope positioning device 36 and laser beam targeting assembly 17 may be secured to different walls of laryngoscope blade 23 or even to different devices entirely.

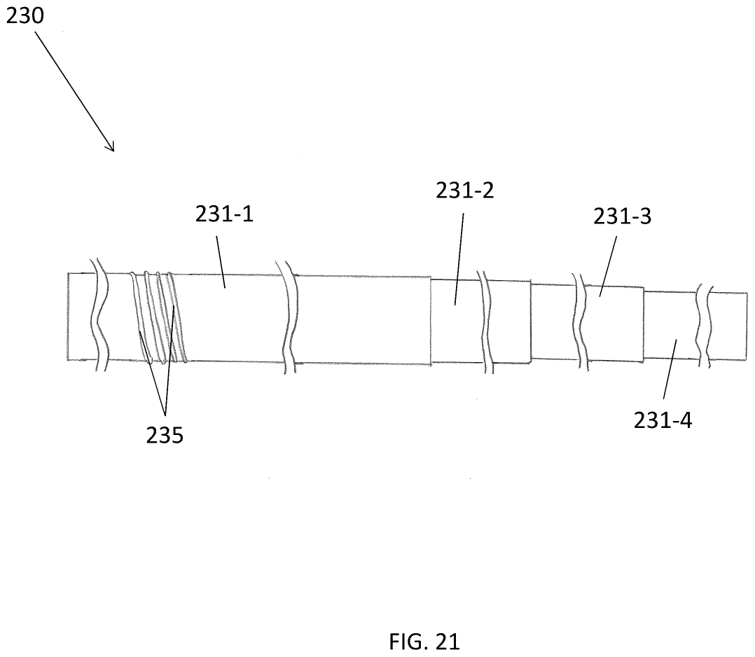

Laser beam targeting assembly 17 may further comprise a wand 230. Wand 230, which is also shown separately in FIG. 21, may comprise a plurality of tubes 231-1 through 231-4, each of which may consist of or comprise a rigid, yet lightweight, material, such as a suitable polymeric or metallic material. Tubes 231-1 through 231-4, which may be generally shaped as concentric cylinders, may be assembled in a conventional fashion to form a telescoping structure of adjustable length. In the present embodiment, wand 230 may be locked, using cam locks or the like, in its most elongated state so that the distal end of wand 230 may be positioned at or beyond distal end 29 of laryngoscope blade 23; however, because it may be desirable, in certain circumstances, for wand 230 to have a shorter length, one or more of tubes 231-1 through 231-4 may be moved axially towards one another and then locked in place to form a structure of shorter length. That being said, where adjustability in length is not needed, wand 230 may consist of a single tube, as opposed to comprising a plurality of telescoping tubes. Tube 231-1 may be provided with an external thread 235, which may be used to matingly engage internal thread 207 on first bore 203, thereby enabling wand 230 to be secured to frame 181.

Laser beam targeting assembly 17 may further comprise a mirror 241. Mirror 241, which may be made of a highly reflective material capable of withstanding irradiation from a surgical laser, may be used to reflect the laser light emitted from the handpiece mounted in second bore 205. In the present embodiment, mirror 241 may be a planar member of generally rectangular shape; however, it is to be understood that mirror 241 need not be planar and need not be of generally rectangular shape. In any event, regardless of its shape, mirror 241 is preferably dimensioned appropriately so as to be positionable in-line with second bore 205.

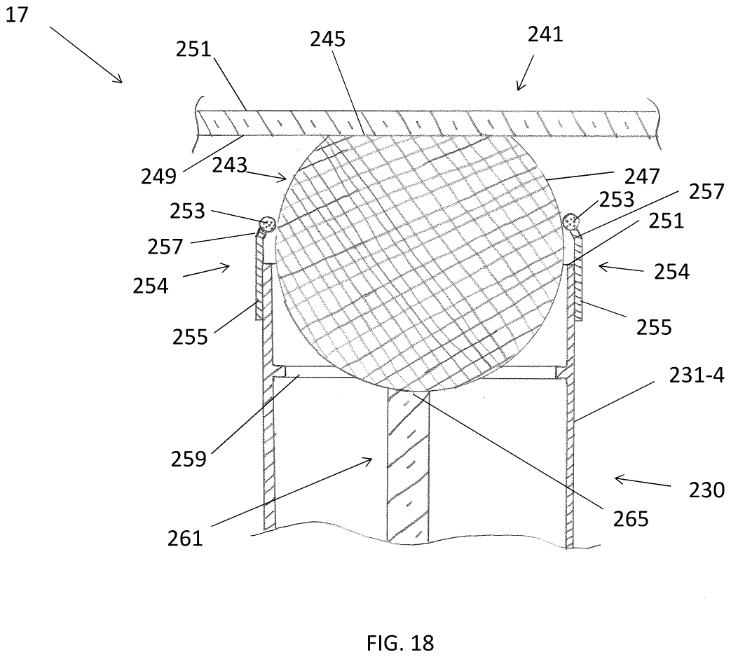

As seen best in FIG. 18, mirror 241 may be coupled to wand 230 using a ball mount 243. In the present embodiment, ball mount 243 may consist of or comprise a rigid, yet lightweight, material, such as a suitable polymeric or metallic material, and may be in the shape of a solid frustospherical structure having a flat surface 245 and an arcuate surface 247. Flat surface 245, which may be rearwardly facing, may be fixedly secured to a front surface 249 of mirror 241. Ball mount 243 may be positioned at a distal end 251 of tube 231-4 and may be retained distally therein by one or more ball bearings 253, each of which may be mounted on a bracket 254 having a first arm 255 fixedly mounted on the exterior of tube 231-4 and a second arm 257 manually deflectable away from or towards ball mount 243. In one embodiment, bracket 254 may be made of spring steel or a similar material. In this manner, one may mount ball mount 243 in tube 231-4 by deflecting second arms 257 radially outwardly from distal end 251 of tube 231-4; then, inserting ball mount 243 proximally into tube 231-4 through distal end 251; and, then, deflecting second arms 257 radially inwardly (or allowing second arms 257 to spring back to their original position) until ball bearings 253 contact ball mount 243. With ball mount 243 mounted in the above-described manner, mirror 241 is capable of being angularly adjusted via three-axis adjustment (i.e., tip, tilt and rotation). Proximal movement of ball mount 243 within tube 231-4 may be constrained by an internal rib 259, which may extend circumferentially within tube 231-4 a short distance from distal end 251. In alternative embodiments, one may utilize hemicylindrical or rectangular configurations for the extension mechanism while maintaining the same functional capabilities.

As can readily be appreciated, alternative arrangements for retaining a structure like ball mount 243 at the distal end of a tube to permit the rotation of said structure over many degrees in any direction or to permit rotational movement of said structure along only certain defined directions are well-known and may be employed instead of the arrangement disclosed above. For example, in another embodiment (not shown), the lower hemisphere of ball mount 243 may be inserted proximally into tube 243-1 through distal end 251. A circumferential rib provided on the interior surface of tube 231-4 at or near distal end 251 may be used to keep ball mount 243 from escaping distally through distal end 251. In addition, an internal rib like internal rib 259 may be used to keep ball mount 243 from moving too far proximally. It should be noted that, in the aforementioned example, tube 243-1 may be constructed of a sufficiently deformable material to permit ball mount 243 to be inserted proximal through distal end 251 and, thereafter, to be retained by the circumferential rib at or near distal end 251.

Laser beam targeting assembly may further comprise a mirror steering mechanism (i.e., a mechanism for adjusting the angular orientation of mirror 241). In the present embodiment, said mirror steering mechanism may comprise a rod 261. Rod 261 may be an elongated member of cylindrical shape consisting of or comprising a rigid, yet lightweight, material, such as a suitable polymeric or metallic material. Rod 261 may have a proximal end 263 and a distal end 265. Rod 261 may be at least partially disposed within wand 230, with proximal end 263 of rod 261 extending a short distance proximally through a proximal end 267 of wand 230 (or being disposed within wand 230 a short distance distally from proximal end 267) and with distal end 265 of rod 261 being fixedly mounted on ball mount 243. Accordingly, by moving distal end 263 of rod 261 in a given direction, ball mount 243 may be caused to rotate in an opposite direction, thereby causing the angular orientation of mirror 241 to be adjusted correspondingly. If needed, a retainer or similar mechanism may be used to maintain rod 261 in a given angular position relative to wand 230. Also, as can readily be appreciated, the ease with which ball mount 243 may rotate within wand 230 and the ease with which rod 261 may be moved within wand 230 may be adjusted so that, once rod 261 has been placed in a desired angular position within wand 230, rod 261 and ball mount 243 will remain stationary until rod 261 is deliberately moved.

It is to be understood that, although a single rod 261 is shown in the present embodiment, one could use two or more such rods to rotate ball mount 243.

Also, as can readily be appreciated, alternative arrangements for rotating a structure like ball mount 243 within a structure like wand 230 may be employed instead of the above-described arrangement. For example, one such alternative arrangement may be a motorized arrangement for rotating ball mount 243, wherein said motorized arrangement may include a joystick used to control, either through a wireless connection or through a wired connection, the direction of rotation of ball mount 243. Such an arrangement may comprise, for example, a gyroscopic control interface coupled to a joystick manipulator located outside the patient and accessible to a surgeon. Such a gyroscopic control interface may enable three-axis adjustment (tip, tilt, and rotation) of mirror 241, providing comprehensive targeting capability and pin-point accuracy.

In one embodiment, such a joystick control mechanism may further integrate precision potentiometers that may enable a surgeon to adjust the response sensitivity of mirror 241. The control interface can be configured for linear or exponential response curves, accommodating surgical preferences and procedural requirements. A central locking mechanism may allow the surgeon to temporarily fix the mirror position during critical portions of the procedure. In one embodiment, one primary potentiometer may monitor and control Y-axis rotation by steering a joystick up and down. In contrast, another can control x-axis rotation of the mirror by steering the joystick left and right. These components can enable surgeons to adjust the mirror sensitively to match procedural requirements, with the extra ability to switch between fine control for precise targeting and broader movements for general positioning.

Overall, the mirror platform's ability to maintain precise alignment during adjustments, combined with its gyroscopic stabilization, may enable targeting accuracy previously unattainable in otolaryngeal laser surgery. The innovative design of the invention eliminates traditional compromises between range of motion and targeting precision, providing stable, accurate laser delivery even to areas beyond direct line of sight.

In addition, a gyroscope-based control mechanism may achieve its unique capabilities through several innovative features. In one embodiment, the concentric cylinder design of wand 230 may incorporate precision-engineered bearing surfaces that maintain perfect concentricity while enabling smooth extension and rotation. The mirror mounting system may utilize micro-gimbal bearings that provide near-frictionless movement while maintaining a rigid position once set. These mechanical elements may work with a roller-ball control interface to create a system that responds to intentional adjustments while remaining immune to unintended movements or vibrations, ensuring consistent beam targeting throughout surgical procedures.

To use laser beam targeting assembly 17, one may secure frame 181 to laryngoscope blade 23 using clip 210; and one may matingly couple second bore 205 to a handpiece that is attached to a surgical laser. The above steps may be performed sequentially in any order or may be performed concurrently. Then, ball mount 243 may be rotated so that the exposed front surface 249 of mirror 241 may be positioned at a desired angle to reflect the beam from the surgical laser. As can be appreciated, the angular positioning of mirror 241 may be adjusted over the course of a given laryngology procedure so that the surgical laser beam may be reflected at different angles. Moreover, if desired, the angular positioning of mirror 241 may be adjusted to an extent such that mirror 241 is no longer in-line with the surgical laser beam, thereby allowing the surgical laser beam to strike a target without previously being deflected. After use, frame 181 may be removed from laryngoscope blade 23 using clip 210; and one may uncouple second bore 205 from a handpiece that is attached to a surgical laser. The above steps may be performed sequentially in any order or may be performed concurrently.

It is to be understood that, although, in the present embodiment, laser beam targeting assembly 17 is disclosed as being used in combination with endoscope assembly 15 in laryngoscope blade 23, both endoscope 15 and laser beam targeting assembly 17 are not limited to being used in this manner. Accordingly, in other embodiments, endoscope assembly 15 and laser beam targeting assembly 17 may be used independently of one another, and, additionally, each may be used independently of a laryngoscope. Thus, the present system may be regarded as encompassing two primary subsystems or devices engineered to function independently or as an integrated unit, providing maximum flexibility for surgical applications. The endoscope positioning device may be used as a foundation for stable visualization while the steerable mirror laser platform may be used to enable precise laser delivery to a surgical site. These devices have been carefully designed to complement each other while maintaining independent functionality when required.

In the above discussion, various embodiments of the invention have been described regarding specific implementation details of a system that includes both endoscope positioning and steerable mirror laser delivery capabilities. The described embodiments include arrangements of clip-on frames, deployable tabs, endoscope sheaths, mirror platforms, and control mechanisms that may vary from implementation to implementation. Such variations may include, but are not limited to, the specific mechanisms for tab deployment, mirror control interfaces, sheath configurations, and mounting arrangements. Accordingly, the specification and drawings should be regarded in an illustrative, rather than a restrictive, sense.

Some desirable features, attributes, aspects, innovations and/or advantages that are applicable to one or more embodiments of the present invention may include one or more of the following:
The present invention permits the docking of an endoscope within a laryngoscope and allows for the fixed positioning of the endoscope at one or more depths along the laryngoscope.
The endoscope may be covered by a sheath (which may be disposable) containing an external projection or bumper. The endoscope may travel within the laryngoscope along a frame/track that guides the endoscope within the laryngoscope. Multiple sets of deployable/retractable tabs or “legs” may be part of the inserted frame such that, when one set of legs is deployed, they may abut the projection of the endoscope sheath and, thereby, prevent further advancement or side-to-side movement of the endoscope. The depth of the endoscope within the laryngoscope may be fixed as the projection of the endoscope sheath cannot pass the legs of the frame.
Fixed positioning of the endoscope may facilitate a consistent view while endoscopic guided laser surgery is performed.
An assistant can easily hold a camera head attached to the endoscope using the frame without the risk of moving the endoscope and interfering with a surgical dissection.
Adjustable fixed positioning allows for enhanced visualization at different depths of the airway (e.g., supraglottis, glottis, subglottis).
Fixed positioning of the endoscope allows interchangeability of different types of endoscopes (e.g., 0 vs. 30 vs. 45 vs. 70 vs. 90 degree) which, in combination, can provide a panoramic view of the airway beyond the line of sight of the surgeon before, during, or after dissection.
An extendable steerable mirror assembly (tip-tilt platform) may be docked onto the laryngoscope, together with an attached laser handpiece.
An extendable wand controlling the mirror may be made of multiple concentric tubes, enabling the depth of the wand within/past the laryngoscope to be fixed at one or more depths.
The angle of the mirror at the end of the wand may be moved by a joystick at the surgeon's end (similar to fine motor control provided by existing micromanipulator technology).
A laser beam may be directed at the steerable mirror positioned at or near the distal end of the laryngoscope in order to have the laser beam deflected to a lesion of interest.
The steerable mirror may allow a laser to be steered to points within direct vision of a surgeon, as well as those points beyond the traditional line of sight, and such points/lesions may be visualized simultaneously with an angled endoscope.
Because the extendable steerable mirror may be docked onto a laryngoscope directly, instead of being guided by a micromanipulator attached to a microscope, there is a decreased risk of significant laser migration from tremor, inadvertent movement of the microscope, or inadvertent movement of an operating table.
In the future, there is potential for AI/machine learning to manipulate the mirror steering to aid in laser targeting. A fixed endoscopic view during a procedure may facilitate computer algorithms to determine (1) coordinates of laser targets, (2) appropriate tip/tilt of steerable mirror to aim at such targets, and (3) efficient tracking from one target to the next. Data from otolaryngeal exams may help build lesion recognition by AI and may help in designing the boundaries of ablation and in adjusting the steerable mirror accordingly.
The endoscope sheath is preferably disposable and may comprise a thin cylinder that surrounds the endoscope circumferentially but does not reach the distal end of the endoscope. The endoscope sheath may be secured proximally to the cubic part of the endoscope where a light post may be connected. A bumper or projection on the sheath may be on a distal portion of the sheath and may be a part of the sheath that abuts the endoscope frame and limits further advancement of the endoscope. Possible configurations may include having the bumper or projection located at the very tip of the sheath or at an intermediate location along the sheath. The bumper or projection, itself, may comprise different constructions (e.g., one, two, or three side brackets vs. a ring), but a construction including two side brackets may be preferred.
The endoscope positioning frame is preferably metallic and reusable and may be sterilized along with other standard surgical equipment. The endoscope frame may comprise (1) a clip-on mechanism to hold the frame at the proximal edge of the laryngoscope; (2) a flat support that runs along, but not beyond, the internal lumen of the laryngoscope and on which there may be (a) one or more sets of tabs or “legs” which may be positioned either horizontally (parallel to/in same plane as the flat support) or vertically (perpendicular to the flat support), and (b) an insertion guide proximally through which the endoscope may be slid to prevent side-to-side motion.
The clip of the endoscope positioning device may comprise two limbs, one of which may travel outside the lumen of the laryngoscope (“outer”) and the other of which may travel within the lumen of the laryngoscope (“inner”). The inner limb may be in continuity with the rest of the endoscope positioning frame. The outer limb may be moved towards or away from the outside of the laryngoscope using a spring or similar biasing mechanism. Manual pressure may be applied to the biasing mechanism to separate the outer limb from the inner limb and to allow for insertion of the laryngoscope between the two limbs. When manual pressure is released, the outer and inner limbs may approximate to each other, clasping the laryngoscope in-between.
The inner limb of the clip-on mechanism of the endoscope positioning device may be in continuity with the flat frame which travels within the lumen of the laryngoscope but does not extend beyond the distal aspect of the laryngoscope. On the proximal end of the frame, there may be a guide/channel which permits the endoscope to slide along the frame in appropriate direction and which prevents side-to-side motion as the endoscope is advanced further within the laryngoscope. The length of the frame may be determined based on the length of the laryngoscope being used (which may vary significantly between pediatric and adult), and the width of the frame may be approximately the width of the endoscope. Selectively deployable sets of tabs or “legs” may be perpendicular to the frame from which they extend. Each set of legs may be positioned either in a flat, non-obstructive (horizontal) position or, when deployed, in an obstructive (vertical) position. Fixation of the legs in the obstructive position is performed to prevent the bumper or projection of the endoscope sheath from passing beyond the obstructive legs. Fixation of the frame legs in an obstructive position can be achieved, for example, by screwing the legs into position prior to inserting the frame into the laryngoscope. Alternatively, the legs could be designed such that a particular set of legs may flip up from the horizontal to vertical position using a spring-loaded mechanism actuated by pressing a button on the frame that is accessible to a surgeon at the proximal end of the device. As a future direction, it is possible that the deployment of a given pair of legs could be programmed and automated such that pressing a button on an attached interface connected to the endoscope would enable the given pair of frame legs to flip from the horizontal to vertical direction. For functional and safety reasons, only one set of legs should be deployed in the vertical/obstructive position at a given time.
The frame of the laser beam targeting assembly is preferably metallic, reusable, and may be sterilized along with other standard surgical equipment. By contrast, the wand and mirror assembly of the laser beam targeting assembly is preferably disposable. A laser handpiece may be inserted into the frame and may be secured in place via mating threads.
The laser beam targeting assembly may comprise a clip to hold the assembly at the proximal edge of a laryngoscope and may further comprise a frame or platform having a first docking port for the extendable steerable mirror and a second docking port for a laser handpiece. The second port may be positioned above the first port or vice versa or the two ports may be positioned side-by-side. The clip may have an ability to clasp a laryngoscope by approximating two limbs, one of which may travel outside the lumen of the laryngoscope (“outer”) and the other of which may travel within the lumen of the laryngoscope (“inner”). The inner limb may be in continuity with the rest of the platform. The outer limb may be moved towards or away from the outside of the laryngoscope via a spring-based biasing mechanism. Manual pressure may be applied to the spring/lever of the biasing mechanism to separate the outer limb from the inner limb and to allow for insertion of the laryngoscope between the two limbs. When manual pressure is released, the outer and inner limbs approximate to each other, clasping the laryngoscope in-between. The docking port for the extendable steerable mirror is of a diameter to accommodate a wand that is inserted through the port. The port has a slight upward ramp such that, when a wand is mounted therein, a mirror deployed at an appropriate length from the port is positioned slightly above the plane of the port. This allows a laser beam (which is running parallel to the luminal direction of the laryngoscope) to impinge the mirror. The wand portion of the steerable mirror assembly may be inserted into the first docking port and may be secured therein using mating threads. The extendable wand may be part of a gyroscope and may comprise telescoping cylinders; alternatively, the extendable wand may be designed using circumferential hemicylinders, rectangles, or structures of other shapes. A mirror is preferably disposed at the distal end of the wand. Preferably, the mirror can be rotated (using, for example, a roller ball mechanism) by a manipulator or other mechanism accessible to the surgeon to enable tip, tilt, and rotation of the mirror. The laser handpiece docking port of the frame may be of diameter to accommodate the laser handpiece. The handpiece may be screwed into place with mating threads. The laser handpiece docking port is preferably level; thus, the laser beam may be emitted in a plane parallel to that of the lumen of the laryngoscope.
The sheath and the frame of the endoscope positioning assembly may be designed to be used together; however, either the sheath or the frame of the endoscope positioning assembly may be used on its own but with no obvious advantage as compared to the current state of the art.
Each of the endoscope positioning device and the laser beam targeting assembly may be used on its own or in combination with the other. For example, the laser beam targeting assembly, even if used without the endoscope positioning device, may still be highly effective/advantageous. In this scenario, one hand of a surgeon may be freely driving the endoscope, and the other hand of the surgeon may be manipulating the laser beam targeting assembly. If both the laser beam targeting assembly and the endoscope positioning device are used simultaneously, this may allow a surgical assistant (or, potentially in the future, a robotic

arm) to maintain the endoscope in a fixed position within the laryngoscope while allowing the surgeon to have two hands free for instrumentation, one of which may be used to navigate the mirror and the other of which may be used for suction, for tissue retraction, or for some other purpose.
Because both the endoscope positioning device and the laser beam targeting assembly use a clip-on mechanism, both devices have the ability to be secured to different parts of the laryngoscope (e.g., the endoscope could be to the right or left or above or below the clipped-on mirror).
Calibration of the system may support accurate laser targeting. The system can be calibrated using alignment guides at various distances and angles. The relative positions of both subsystems may be optimized by measuring laser spot positioning at known target points and verifying endoscope visualization. Common calibration procedures utilize standard targeting plates with precise measurement markers.
The system may enable continuous monitoring of both visualization and laser positioning during procedures. A fixed endoscope position may ensure stable imaging while the steerable mirror may allow dynamic adjustment of laser targeting without disturbing visualization. This dual-stability approach may enable precise surgical intervention, even in anatomically challenging regions.
The system can support various surgical approaches based on specific procedural requirements. For example, it can facilitate sequential laser application to multiple targets while maintaining a single stable viewing position, or it can allow repositioning of both visualization and targeting elements for different surgical phases. Standardized positioning mechanisms and control interfaces may ensure consistency across different procedures and operators.
The system may incorporate certain safety features. For example, the deployable stops may include positive locking mechanisms that prevent unintended retraction during use. The mirror control system may incorporate mechanical stops to prevent over-rotation. These safety systems may work in concert to prevent unintended movement or laser misalignment during surgical procedures.
The geometric relationships between components may be carefully engineered to maintain system stability. For example, the projection of the endoscope sheath may be designed to engage with the deployable stops at points that create a three-point contact system, preventing both rotational and linear movement once positioned. The mirror platform's extension mechanism may maintain a constant centerline despite its articulation, ensuring predictable laser targeting throughout its range of motion.
The assembly and calibration of the system may follow a precise protocol. For example, the clips may be designed with alignment markers to ensure proper orientation during mounting. During installation, the deployable stops may undergo a verification sequence to confirm proper deployment force and positioning. The mirror control system may include calibration reference points that allow quick alignment verification during setup. This attention to detail in the assembly process may contribute significantly to the overall accuracy and reliability of the surgical system.
The design of the system may prioritize efficient reprocessing and maintenance. For example, reusable components may be constructed to withstand standard sterilization procedures while disposable elements, such as the endoscope sheath and the extendable mirrors, may be designed for quick replacement without compromising system alignment. Access points for cleaning and inspection may be strategically placed to enable thorough maintenance while preserving the precision of critical components.
The present invention can provide stable endoscope positioning, precise laser targeting beyond direct line of sight, and enhanced surgical access to challenging anatomical regions while reducing operator fatigue and improving procedural efficiency.

The embodiments of the present invention described above are intended to be merely exemplary and those skilled in the art shall be able to make numerous variations and modifications to it without departing from the spirit of the present invention. All such variations and modifications are intended to be within the scope of the present invention as defined in the appended claims.

What is claimed is:

1. An endoscope positioning device, the endoscope positioning device comprising:
(a) a frame, the frame being removably mountable on a proximal end of a laryngoscope blade;
(b) a sheath, the sheath being disposed on the frame and being insertable over a portion of an endoscope; and
(c) wherein the frame and the sheath comprise complementary means for coupling the sheath to the frame in a manner in which distal movement of the sheath relative to the frame is delimited.

2. The endoscope positioning device as claimed in claim 1 wherein the sheath comprises a jacket, the jacket being insertable over a portion of an endoscope insertion tube, wherein the frame comprises a support, the sheath being disposed on the support, and wherein the complementary means comprises a projection extending outwardly from the sheath and a first stop coupled to the support, wherein the first stop is configured to engage the projection.

3. The endoscope positioning device as claimed in claim 1 wherein the sheath comprises a jacket, the jacket being insertable over a portion of an endoscope insertion tube, wherein the frame comprises a support, the sheath being disposed on the support, and wherein the complementary means comprises a projection extending outwardly from the sheath and first and second stops coupled to the support, wherein the first and second stops are coupled to the support at different points along a length of the support, and wherein one of the first and second stops is configured to engage the projection.

4. The endoscope positioning device as claimed in claim 1 wherein the frame comprises a guide, the guide being disposed on the support and aligned therewith, the guide receiving a portion of the sheath.

5. The endoscope positioning device as claimed in claim 1 wherein the frame comprises a clip, the clip being configured to be detachably secured to the proximal end of a laryngoscope blade.

6. The endoscope positioning device as claimed in claim 1 wherein the sheath is dimensioned to delimit axial movement of the endoscope relative to the sheath.

7. An endoscope assembly, the endoscope assembly comprising the endoscope positioning device as claimed in claim 1 and an endoscope, wherein the sheath of the endoscope positioning device is removably inserted over a portion of the endoscope.

8. A system suitable for use in performing an otolaryngology procedure, the system comprising a laryngoscope and the endoscope assembly as claimed in claim 7, wherein the laryngoscope comprises a blade having a channel, and wherein the endoscope assembly is removably mounted on the blade of the laryngoscope, with the endoscope inserted into the channel.

9. The system as claimed in claim 8 further comprising a laser beam targeting assembly, wherein the laser beam targeting assembly is removably mounted on the blade of the laryngoscope.

10. The system as claimed in claim 9 wherein the laser beam targeting assembly comprises a platform, wherein the platform is disposed in the channel, wherein the platform comprises a first docking port, wherein the first docking port is configured to receive a surgical laser handpiece in an orientation to direct a laser beam emitted from the surgical laser handpiece distally through the channel.

11. The system as claimed in claim 10 wherein the platform further comprises a second docking port and wherein the laser beam targeting assembly further comprises an angularly deflectable mirror coupled to the second docking port, the angularly deflectable mirror being configured to reflect the laser beam emitted from the surgical laser.

12. A kit suitable for use in performing an otolaryngology procedure, the kit comprising:
(a) a first frame, the first frame being removably mountable on a proximal end of a laryngoscope blade;
(b) a sheath, the sheath being positionable on the first frame and being insertable over a portion of an endoscope; and
(c) wherein the first frame and the sheath comprise first complementary means for coupling the sheath to the first frame in a manner in which distal movement of the sheath relative to the first frame is delimited.

13. The kit as claimed in claim 12 wherein the sheath comprises a jacket, the jacket being insertable over a portion of an endoscope insertion tube, wherein the first frame comprises a first support, the sheath being positionable on the first support, and wherein the first complementary means comprises a projection extending outwardly from the sheath and a first stop coupled to the first support, wherein the first stop is engageable with the projection.

14. The kit as claimed in claim 12 wherein the sheath comprises a jacket, the jacket being insertable over a portion of an endoscope insertion tube, wherein the first frame comprises a first support, the sheath being positionable on the first support, wherein the first complementary means comprises a projection extending outwardly from the sheath and first and second stops coupled to the first support, wherein the first and second stops are positioned at different points along a length of the first support, and wherein the first and second stops are alternatively engageable with the projection.

15. The kit as claimed in claim 14 wherein each of the first and second stops is transformable between a deployed state engageable with the projection and a non-deployed state not engageable with the projection.

16. The kit as claimed in claim 15 wherein each of the first and second stops is transformable by being bent manually.

17. The kit as claimed in claim 12 further comprising a second frame, the second frame being removably mountable on the proximal end of the laryngoscope blade, wherein the second frame and the sheath comprise second complementary means for coupling the sheath to the second frame in a manner in which distal movement of the sheath relative to the second frame is delimited, and wherein the first frame and the second frame are configured to position a distal end of an endoscope at different depths in the laryngoscope blade.

18. The kit as claimed in claim 12 further comprising a laser beam targeting assembly, wherein the laser beam targeting assembly comprises a platform removably mountable in a laryngoscope blade, wherein the platform comprises a first docking port, wherein the first docking port is configured to receive a surgical laser handpiece in an orientation to direct a laser beam emitted from the surgical laser handpiece distally through the channel.

19. The kit as claimed in claim 18 wherein the platform further comprises a second docking port and wherein the laser beam targeting assembly further comprises an angularly deflectable mirror coupled to the second docking port, the angularly deflectable mirror being configured to reflect the laser beam emitted from the surgical laser.

20. The kit as claimed in claim 19 wherein the laser beam targeting assembly further comprises a ball mount and a wand, the ball mount and the wand coupling the angularly deflectable mirror to the second docking port.
